(19) United States
(12) Patent Application Publication
(10) Pub. No.: US 2019/0004353 A1
(43) Pub. Date: Jan. 3, 2019

(54) Systems, Devices, and Methods for Managing Data

(71) Applicant: METER Group, Inc. USA, Pullman, WA (US)

(72) Inventor: Scott H. Campbell, Pullman, WA (US)

(73) Assignee: METER Group, Inc. USA, Pullman, WA (US)

(21) Appl. No.: 15/640,391

(22) Filed: Jun. 30, 2017

(51) Int. Cl. G02F 1/1333; G06F 3/041; F16M 11/10; F16M 13/02; G01N 22/04; G01N 35/00; G01N 33/18

(52) U.S. Cl. CPC G02F 1/13338; G06F 3/0416; F16M 11/10; G01N 33/18; G01N 22/04; G01N 35/00871; F16M 13/02

(57) ABSTRACT

Apparatus, devices, systems, and techniques are disclosed herein to manage digital data generated as part of a manufacturing process. A data management device may be operatively coupled to one or more instruments via cables connected to data ports provided in the instruments. The data management device may be configured to communicate with the instruments via these wired connections. The data management device may be network-enabled such that it is capable of communicating with servers and/or remote computing devices via a network. Upon executing a test, the data management device may collect the data from the instruments and form a digital record. The digital record may include measurement information and compliance information associated with the test that was performed. The data management device may be configured to store the digital record or the test data.

### BACKGROUND

The following relates generally to systems, devices, and methods for managing data.

Manufacturing processes generally and food manufacturing processes more specifically have been regulated in most industrialized nations for many years. For example, in the United States the Food and Drug Administration (FDA) and/or the United States Department of Agriculture (USDA) sets and enforces rules and regulations related to the production of food.

To ensure public safety, these food regulators have set quality standards for food that must be met by food manufacturers as well as documentation and reporting requirements for those food manufacturers. For example, a food regulator may require that a certain number of food samples in a food production process be tested during the process. These regulators may require that such samples have certain characteristics before the food produced can be sold to the public. In practice, the FDA may require that a food manufacturing plant meet certain standards prior to allowing the plant to produce food that is sold to the public.

To comply with such regulations and standards, food manufacturers may establish workflows, best practices, or other procedures to ensure products comply with the relevant standards. In addition, food manufactures may also establish workflows, best practices, or other procedures to meet the documentation and reporting standards associated with the relevant standards. Such testing and documentation frequently produces hand-written lab notes and paper documentation. When a regulating body audits a food manufacturer, the food manufacturer may produce the paper records to show compliance with the relevant standards or regulations.

### SUMMARY

In one embodiment, a device may include a pillar extending upward from a base, a plurality of data ports positioned on a first sidewall of the pillar, each data port may be configured to receive data output from a serial port of a water activity meter, and a touchscreen user interface coupled to a second sidewall of the pillar different from the first sidewall, the touchscreen user interface may be angled relative to the first sidewall such that the touchscreen user interface may define a viewing angle, the touchscreen user interface may be configured to output the received data and received commands from a user.

Some examples of the device described above may also include an arm extending from the second sidewall of the pillar configured to suspend the touchscreen user interface above a top surface of the base, wherein the touchscreen user interface may be coupled to the arm.

In some examples of the device described above, the arm may be angled relative to the first sidewall of the pillar.

Some examples of the device described above may also include a first distance between a first end of the arm and the first sidewall may be less than a second distance between a second end of the arm and the first sidewall.

In some examples of the device described above, the pillar and the touchscreen user interface may be positioned completely above the base such that side surfaces of the base may be positioned beyond the pillar and the touchscreen user interface.

In some examples of the device described above, the base may comprise a top surface having a first base dimension extending in a first direction greater than a second base dimension extending in a second direction of the top surface perpendicular to the first direction, and the pillar may comprise a top wall having a first pillar dimension extending in the first direction that may be less than a second pillar dimension extending in the second direction.

In some examples of the device described above, the touchscreen user interface may extend from a medial surface spaced apart from the second sidewall to a lateral surface positioned between the second sidewall and a first side surface of the base.

In some examples of the device described above, the base may comprise a top surface extending from a first side surface to a second side surface positioned opposite the first side surface, and the pillar may be positioned on the base such that a first distance between the second sidewall of the pillar and the first side surface of the base may be greater than a second distance between a third sidewall of the pillar and the second side surface of the base, wherein the third sidewall may be positioned opposite the second sidewall.

Some examples of the device described above may also include a third distance between the second sidewall and a lateral surface of the touchscreen user interface may be less than the first distance.

In some examples of the device described above, the base further may comprise a third side surface extending between the first side surface and the second side surface and a fourth side surface extending between the first side surface and the second side surface, the fourth side surface may be positioned opposite the third side surface, and a fourth distance between the first sidewall and the third side surface of the base may be less than a fifth distance between a superior surface of the touchscreen user interface and the third side surface of the base.

Some examples of the device described above may also include a sixth distance between a fourth sidewall of the pillar positioned opposite the first sidewall and the fourth side surface of the base may be greater than a seventh distance between an inferior surface of the touchscreen user interface and the fourth side surface of the base.

In some examples of the device described above, the viewing angle of the touchscreen user interface may be non-orthogonal relative to a top surface of the base.

In some examples of the device described above, the touchscreen user interface may extend above a top wall of the pillar.

In some examples of the device described above, the touchscreen user interface may define a lateral surface positioned between the second sidewall of the pillar and a first side surface of the base.

In some examples of the device described above, the touchscreen user interface may include a housing and a screen.

Some examples of the device described above may also include a processor and a memory configured to communicate data with the water activity meter via at least one of the plurality of data ports.

In some examples of the device described above, the water activity meter may be configured to measure a water activity of a food sample in a food manufacturing process.

In some examples of the device described above, the plurality of data ports may be a plurality of universal serial bus (USB) ports.

In one embodiment, a system may include an instrument configured to measure a parameter of a food sample in a food manufacturing process, the instrument having a serial port configured to output data indicative of the parameter, a data management device including: a pillar extending upward from a base, a plurality of universal serial data (USB) ports positioned on a first sidewall of the pillar, each USB data port may be configured to receive the data output from the serial port of the instrument, and a touchscreen user interface coupled to a second sidewall of the pillar different from the first sidewall, the touchscreen user interface may be angled relative to the first sidewall such that the touchscreen user interface may define a viewing angle, the touchscreen user interface may be configured to output the received data and receive commands from a user.

Some examples of the system described above may also include a cable having a USB cable connector at a first end and a serial cable connector at a second, the cable extending between one of the USB ports of the data management device and the serial port of the instrument.

In some examples of the system described above, the instrument may include a first sensor to measure the parameter of the food sample and a second sensor to identify a position of the food sample relative to the first sensor.

In some examples of the system described above, the instrument may be a water activity meter and the parameter may be a water activity.

In some examples of the system described above, the instrument may be a moisture analyzer and the parameter may be moisture parameter.

A method for managing data is described. The method may include receiving, by a data management device, instructions to test a characteristic of a food sample in a food manufacturing process, identifying a water activity meter from a plurality of water activity meters connected to the data management device to perform the test of the characteristic based at least in part on validation data associated with each water activity meter of the plurality of water activity meters, receiving proximity data from the identified water activity meter indicating that a first sensor of the identified water activity meter is positioned in a predefined location relative to the food sample, and transmitting a message to the identified water activity meter to initiate the test based at least in part on receiving the proximity data.

An apparatus for managing data is described. The apparatus may include means for receiving, by a data management device, instructions to test a characteristic of a food sample in a food manufacturing process, means for identifying a water activity meter from a plurality of water activity meters connected to the data management device to perform the test of the characteristic based at least in part on validation data associated with each water activity meter of the plurality of water activity meters, means for receiving proximity data from the identified water activity meter indicating that a first sensor of the identified water activity meter is positioned in a predefined location relative to the food sample, and means for transmitting a message to the identified water activity meter to initiate the test based at least in part on receiving the proximity data.

Another apparatus for managing data is described. The apparatus may include a processor, memory in electronic communication with the processor, and instructions stored in the memory. The instructions may be operable to cause the processor to receive, by a data management device, instructions to test a characteristic of a food sample in a food manufacturing process, identify a water activity meter from a plurality of water activity meters connected to the data management device to perform the test of the characteristic based at least in part on validation data associated with each water activity meter of the plurality of water activity meters, receive proximity data from the identified water activity meter indicating that a first sensor of the identified water activity meter is positioned in a predefined location relative to the food sample, and transmit a message to the identified water activity meter to initiate the test based at least in part on receiving the proximity data.

A non-transitory computer readable medium for managing data is described. The non-transitory computer-readable medium may include instructions operable to cause a processor to receive, by a data management device, instructions to test a characteristic of a food sample in a food manufacturing process, identify a water activity meter from a plurality of water activity meters connected to the data management device to perform the test of the characteristic based at least in part on validation data associated with each water activity meter of the plurality of water activity meters, receive proximity data from the identified water activity meter indicating that a first sensor of the identified water activity meter is positioned in a predefined location relative to the food sample, and transmit a message to the identified water activity meter to initiate the test based at least in part on receiving the proximity data.

Some examples of the method, apparatus, and non-transitory computer-readable medium described above may further include processes, features, means, or instructions for determining a position of the food sample relative to the first sensor based at least in part on the proximity data received from a second sensor associated with the identified water activity meter.

Some examples of the method, apparatus, and non-transitory computer-readable medium described above may further include processes, features, means, or instructions for receiving, from the first sensor of the identified water activity meter, sensor data indicating a parameter of the food sample based at least in part on the water activity meter performing the test.

Some examples of the method, apparatus, and non-transitory computer-readable medium described above may further include processes, features, means, or instructions for outputting the sensor data via a touchscreen user interface of the data management device.

Some examples of the method, apparatus, and non-transitory computer-readable medium described above may further include processes, features, means, or instructions for outputting an indication of the proximity data via a touchscreen user interface of the data management device.

Some examples of the method, apparatus, and non-transitory computer-readable medium described above may further include processes, features, means, or instructions for receiving an input command from a user to initiate the test via the touchscreen user interface based at least in part on outputting the indication.

Some examples of the method, apparatus, and non-transitory computer-readable medium described above may further include processes, features, means, or instructions for comparing the validation data of each water activity meter, wherein identifying the water activity meter may be based at least in part on the comparison.

In some examples of the method, apparatus, and non-transitory computer-readable medium described above, the validation data may comprise information indicating whether a validation procedure associated with the water activity meter may have been implemented.

In some examples of the method, apparatus, and non-transitory computer-readable medium described above, the validation data comprising information indicating a maintenance report associated with the water activity meter and a calibration report associated with the water activity meter.

A method of managing data is described. The method may include receiving, by a data management device, sensor data from a water activity meter based on a test performed by the water activity meter, the sensor data may be indicative of a water activity present in a food sample taken during a food manufacturing process, identifying criterion associated with the water activity based at least in part on a mode of operation of the data management device, comparing the sensor data to a threshold specified by the criterion, and outputting an indication based at least in part on the sensor data failing to satisfy the threshold.

An apparatus for managing data is described. The apparatus may include means for receiving, by a data management device, sensor data from a water activity meter based on a test performed by the water activity meter, the sensor data may be indicative of a water activity present in a food sample taken during a food manufacturing process, means for identifying criterion associated with the water activity based at least in part on a mode of operation of the data management device, means for comparing the sensor data to a threshold specified by the criterion, and means for outputting an indication based at least in part on the sensor data failing to satisfy the threshold.

Another apparatus for managing data is described. The apparatus may include a processor, memory in electronic communication with the processor, and instructions stored in the memory. The instructions may be operable to cause the processor to receive, by a data management device, sensor data from a water activity meter based on a test performed by the water activity meter, the sensor data may be indicative of a water activity present in a food sample taken during a food manufacturing process, identify criterion associated with the water activity based at least in part on a mode of operation of the data management device, compare the sensor data to a threshold specified by the criterion, and output an indication based at least in part on the sensor data failing to satisfy the threshold.

A non-transitory computer readable medium for managing data is described. The non-transitory computer-readable medium may include instructions operable to cause a processor to receive, by a data management device, sensor data from a water activity meter based on a test performed by the water activity meter, the sensor data may be indicative of a water activity present in a food sample taken during a food manufacturing process, identify criterion associated with the water activity based at least in part on a mode of operation of the data management device, compare the sensor data to a threshold specified by the criterion, and output an indication based at least in part on the sensor data failing to satisfy the threshold.

Some examples of the method, apparatus, and non-transitory computer-readable medium described above may further include processes, features, means, or instructions for transmitting, automatically, a message to the water activity meter to perform another test based at least in part on the sensor data failing to satisfy the threshold.

Some examples of the method, apparatus, and non-transitory computer-readable medium described above may further include processes, features, means, or instructions for identifying that a number of failed tests does not satisfy a test threshold, wherein transmitting the message may be based at least in part on the test threshold not may be satisfied.

Some examples of the method, apparatus, and non-transitory computer-readable medium described above may further include processes, features, means, or instructions for receiving an input command from a user via a touchscreen user interface of the data management device based at least in part on outputting the indication.

In some examples of the method, apparatus, and non-transitory computer-readable medium described above, the data management device may include a first mode of operation specifying a first set of criteria associated with the water activity and a second mode of operation specifying a second set of criteria associated with the water activity, the second set of criteria may be more strict than the first set of criteria.

### BRIEF DESCRIPTION OF THE DRAWINGS

The accompanying drawings and figures illustrate a number of exemplary embodiments and are part of the specification. Together with the present description, these drawings demonstrate and explain various principles of this disclosure. A further understanding of the nature and advantages of the present invention may be realized by reference to the following drawings. In the appended figures, similar components or features may have the same reference label.

FIG. 1 illustrates a block diagram showing a lab system according to an embodiment of the present disclosure.

FIG. 2 illustrates a perspective view of an embodiment of an apparatus of the present disclosure.

FIG. 3 illustrates a front elevation view of the apparatus of FIG. 2.

FIG. 4 illustrates a side elevation view of the apparatus of the apparatus of FIG. 2.

FIG. 5 illustrates a rear elevation view of the apparatus of FIG. 2.

FIG. 6 illustrates a block diagram of the apparatus of FIG. 2.

FIG. 7 illustrates a block diagram of an embodiment of an instrument of the present disclosure.

FIG. 8 illustrates an embodiment of a communication scheme of the lab system of FIG. 1.

FIG. 9 illustrates an embodiment of a method by which the lab system of FIG. 1 operates.

FIG. 10 illustrates an embodiment of a method by the apparatus of FIG. 2 operates.

While the embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the instant disclosure covers all modifications, equivalents, and alternatives falling within the scope of the appended claims.

### DETAILED DESCRIPTION

When documenting compliance with regulations and other requirements, a manufacturer may rely on paper records to show that regulations have been met. For example, in food manufacturing, a manufacturer may test a prescribed number of food samples from each batch of food manufactured to determine whether the batch complies with certain standards. In addition, the manufacturer may be required to document each test performed on each sample.

Keeping such records on paper files may introduce human error to the record-keeping procedure. For example, transcription errors may be introduced as a human reads a measurement from a device and subsequently records it in a lab notebook. In another example, a human may miss certain details prior to performing a test, thereby rendering the results invalid (e.g., the human may use a non-compliant instrument to perform the test).

Moreover, many of the instruments used to perform such tests may not be capable interacting with one or more communication networks that are have become more common place in recent years (e.g., Wi-Fi, WLAN, LTE). The instruments may not be capable of communicating test data and sensor data in a digital form to other computing devices unaided.

Apparatus, devices, systems, and techniques are disclosed herein to manage digital data generated as part of a manufacturing process. The digital data generated as part of a manufacturing process may include lab data, test data, sensor data, other types of data described herein, or combinations thereof. A data management device may be operatively coupled to one or more instruments via cables connected to data ports provided in the instruments. The data management device may be configured to communicate with the instruments via these wired connections. The data management device may be network-enabled such that it is capable of communicating with servers and/or remote computing devices via a network. Upon executing a test, the data management device may collect the data from the instruments and form a digital record. The digital record may include measurement information and compliance information associated with the test that was performed. The data management device may be configured to store the digital record or the test data.

The systems, devices, and techniques described provide a variety of advantages. For example, a system for generating and storing digital records may remove several events that may introduce human error into a record keeping process (e.g., transcription errors). In other examples, the systems, devices, and techniques described may ensure a higher quality of test results by providing quality assurance checks prior to performing a test. In other examples, the systems, devices, and techniques may more reliably store such digital records without information being lost. These advantages are presented as examples only, the systems, devices, and techniques also may provide other advantages that are apparent to a person of ordinary skill in the art.

The following description provides examples and is not limiting of the scope, applicability, and/or examples set forth in the claims. Changes may be made in the function and/or arrangement of elements discussed without departing from the scope of the disclosure. Various examples may omit, substitute, and/or add various procedures and/or components as appropriate. For instance, the methods described may be performed in an order different from that described, and/or various steps may be added, omitted, and/or combined. Also, features described with respect to some examples may be combined in other examples.

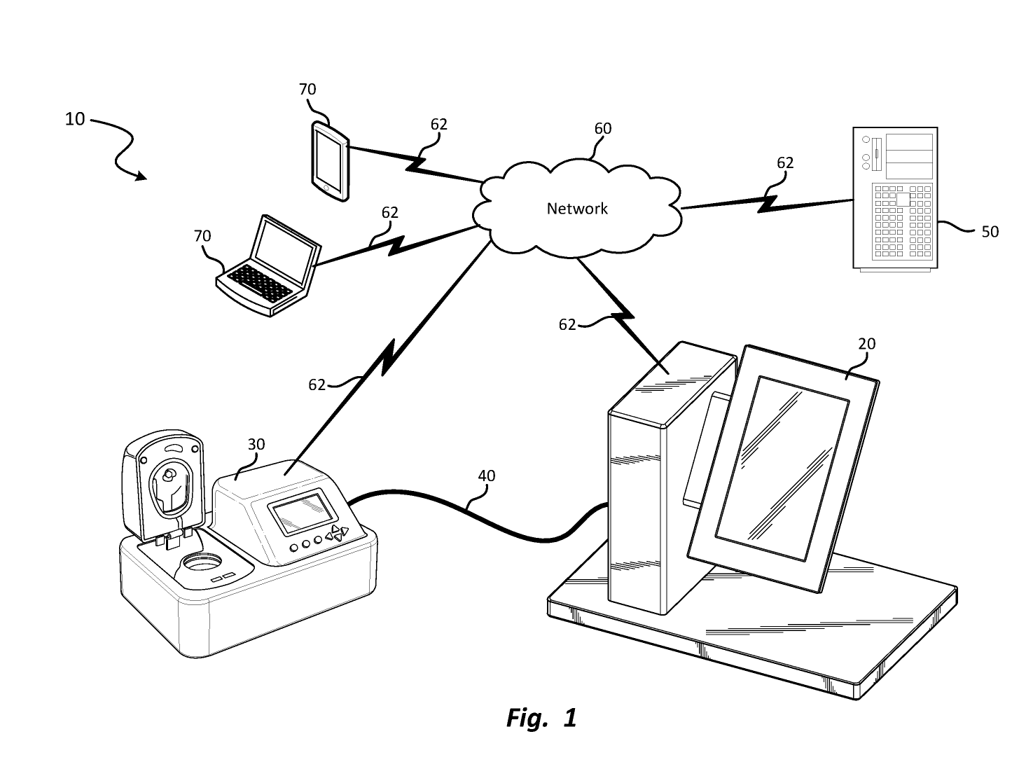

FIG. 1 illustrates a lab system 10 configured to test samples taken as part of a manufacturing process. In some examples, the lab system 10 may be configured to test food samples taken in a food manufacturing process. In other examples, the lab system 10 may be configured to test other types of samples that include water or moisture. In some examples, the lab system 10 may be implemented in a setting other than a lab of a food manufacturing process.

The lab system 10 may comprise a data management device 20, one or more instruments 30 including one or more sensors configured to measure characteristics of the sample, and a cable 40 for operatively coupling the data management device 20 and the instrument 30. In some examples, the lab system 10 also may include a server 50, a network 60, and one or more remote computing device(s) 70. The server 50 may communicate with the data management device 20 and the instrument 30 via the network 60. The remote computing devices 70 may communicate with any of the other entities of the lab system 10 via the network 60, in some examples. In some cases, the data management device 20 may be a lab data management device and the instrument 30 may be a lab instrument.

The lab system 10 may be an example of a centralized data management system configured to allow data to be stored, managed, or controlled in a digital manner. The types of data that may be stored, managed, or controlled in a digital manner may include lab data, sample data, manufacturing data, sensor data, instrument data, user data, test data, authentication data, quality control data, quality assurance data, validation data, other types of data, or combination thereof. In such a system, a user may interact with the lab entities (or data generated therefrom) via the data management device 20 or the remote computing devices 70. In addition, the data management device 20 may generate digital records (e.g., digital lab records) based on data and store them on the server 50.

The data management device 20 may be configured to collect data from multiple sources and create digital records based on the data collected. The data management device 20 may be configured to be operatively coupled to legacy lab instruments and to the network 60. As used herein, a legacy device may be a device that is not enabled to access the network 60. For example, a legacy device may not include a wireless network interface, in some instances. In some examples, a lab system 10 may include more than one data management devices 20. The data management device 20 may be a dedicated computing entity configured to be operated in a laboratory.

The data management device 20 also may be configured to manage and/or operate one or more instruments 30 at a time. The data management device 20 may perform workflows (e.g., lab workflows) using one or more connected instruments 30. The data management device 20 may convert data into a digital record. In some examples, the data management device 20 may transmit the digital record and/or the data to the server 50 to be stored. The data management device 20 may be configured to perform quality assurance checks on data and workflows. The data management device 20 may verify digital reports, generate certificates of analysis, monitor system success, document data, or combinations thereof. The data management device 20 may be configured to transmit data, messages, commands, or combinations thereof to one or more instruments 30. The data management device 20 may be configured to receive data, messages, commands, or combinations thereof from the one or more instruments 30. In some examples, the data management device 20 may enable a legacy instrument 30 to access the network 60 and thereby communicate with other entities of the lab system 10 (e.g., server 50, remote computing devices 70).

The instruments 30 may be configured to measure one or more characteristics of a sample. In some examples, the instruments 30 may be a testing device found in a lab. In some examples, the instrument 30 may be a water activity meter configured to measure the water activity of a sample. In some examples, the instrument 30 may be a moisture analyzer configured to measure the moisture content of a sample. While only one instrument 30 is depicted in FIG. 1, the data management device 20 may be operatively coupled to multiple instruments 30 via either a plurality of cables 40 or the network 60. The instrument 30 may include a housing, a container to accept a sample, a user interface, and a plurality of sensors to perform a variety of tasks.

The instrument 30 may be any type of instrument or lab instrument. For example, an instrument 30 may include a pH meter, a moisture analyzer, a water activity detector, a near infrared (NIR) spectrometer, a color analyzer, a salt analyzer, a temperature analyzer, an oxygen analyzer, a weight measurement device (e.g., a balance), or combinations thereof. Each of the different types of instruments 30 may also include their relevant sensors for detecting a measuring their respective characteristics.

The instrument 30 may be operatively coupled to the data management device 20 via one or more cables 40. The cable 40 may include a serial connector configured to mate with a serial port of the instrument 30 and a universal serial bus (USB) connector configured to mate with a USB port of the data management device 20. In some examples, the data management device 20 may be connected to the instrument(s) 30 via some other type of communication link.

The cable 40 may operatively couple the data management device 20 to an instrument 30. Some legacy lab instruments may not be enabled to access wireless networks (e.g., network 60). Rather, these legacy lab instruments may be stand-alone devices that are configured to be accessed by users physically in the lab where the legacy lab instrument is located. These legacy lab instruments may include only a limited number and a limited type of data ports to communicate with other devices. The cable 40 may be configured to operatively couple an instrument with a first connection type to a data management device with a second connection type.

The cable 40 may include a first connector configured to mate with a data connection on the data management device 20. For example, the first connector of the cable 40 may be a USB connector configured to mate with a USB port on the data management device 20. The cable 40 may also include a second connector configured to mate with a data connection on the instruments. For example, the second connector of the cable 40 may be a serial connector, as understood by those skilled in the art, configured to mate with a serial port on the instrument 30. The first and second connectors of the cable 40 may be connectors utilizing the following standards or technology: video graphics array (VGA), digital visual interface (DVI), high definition multimedia interface (HDMI), integrated drive electronics (IDE), serial advanced technology attachment (SATA), external serial advanced technology attachment (eSATA), FireWire, Ethernet, PS/2, serial connections, RS-232 serial, DB-25 serial, DE-9 serial, S-Video, DIN, or combinations thereof.

The server 50 may be configured to facilitate communication between the data management device 20 and the remote computing device(s) 70. The server 50 may be configured to store data and/or reports received from the data management device 20 or the instrument(s) 30. The server 50 may be configured to communicate data, messages, commands, or combinations thereof between the data management device 20 and the remote computing device(s) 70. The data, messages, or commands may originate at either the data management device 20 or the remote computing device(s) 70.

The server 50 may be configured to communicate with the data management device 20, the instruments 30, or the remote computing devices 70. The server 50 may perform additional processing on signals, data, messages, or commands received from the various entities of the lab system 10, or the server 50 may simply forward the received signals, data, messages, or commands to various entities in the lab system 10.

The server 50 may be a computing device operable to receive data streams (e.g., from data management device 20, instruments 30, or remote computing devices 70), store and/or process data, and/or transmit data. For example, server 50 may receive sensor data, validation data, encryption data, metadata associated with the sensor data, instrument data, workflow data, sample data, testing data, or various combinations thereof from the data management device 20, the instruments 30, the remote computing devices 70, or various combinations thereof. In some embodiments, server 50 may “pull” the data from the other entities, e.g., by querying the other entities in the lab system 10 (e.g., data management device 20, instruments 30, remote computing devices 70). In some embodiments, the data streams may be “pushed” by other entities in the lab system 10 (e.g., data management device 20, instruments 30, remote computing devices 70) to the server 50. For example, the data management device 20, instruments 30, or remote computing devices 70 may be configured to transmit data as it is generated by or entered into that device. In some instances, the other entities in the lab system 10 may periodically transmit data (e.g., as a block of data or as one or more data points) without being queried.

The server 50 may include a database (e.g., in memory) containing sensor data, validation data, encryption data, metadata associated with the sensor data, instrument data, workflow data, sample data, testing data, or various combinations thereof received from the other entities of the lab system 10. Additionally, software (e.g., stored in memory) may be executed on a processor of the server 50. Such software (executed on the processor) may be operable to cause the server 50 to monitor, process, summarize, present, and/or send a signal associated with resource usage data.

The network 60 may communicate data, messages, or commands via wired or wireless communication links 62 between entities of the lab system 10. In alternate embodiments, the network 60 may be integrated with any one of the data management device 20, the instruments 30, the server 50, or the remote computing devices 70, such that separate components are not required. The network 60 may be the Internet, an intranet, a personal area network, a local area network (LAN), a wide area network (WAN), a virtual network, a telecommunications network implemented as a wired network and/or wireless network, etc.

The remote computing devices 70 may be configured to allow a user to interact with the lab system 10. As used herein, a remote computing device 70 may be an example of a computing device that communicates with data management device 20 or the instruments 30 via the server 50. In some examples, the remote computing devices 70 may not establish direct wireless communication links 62 with either the data management device 20 or the instruments 30.

The remote computing device(s) 70 may be custom computing entities configured to interact with the data management device 20, the instruments 30, the server 50, or various combinations thereof via the network 60. In other examples, the remote computing device(s) 70 may communicate with some entities of the lab system 10 via the server 50. The remote computing device(s) 70 may be general purpose computing entities such as a personal computing device, for example, a desktop computer, a laptop computer, a netbook, a tablet personal computer (PC), a control panel, an indicator panel, a multi-site dashboard, an iPod®, an iPad®, a smart phone, a mobile phone, a personal digital assistant (PDA), and/or any other suitable device operable to send and receive signals, store and retrieve data, and/or execute modules.

The remote computing device(s) 70 may include memory, a processor, an output, a data input and a communication module. The processor may be a general purpose processor, a Field Programmable Gate Array (FPGA), an Application Specific Integrated Circuit (ASIC), a Digital Signal Processor (DSP), and/or the like. The processor may be configured to retrieve data from and/or write data to the memory. The memory may be, for example, a random access memory (RAM), a memory buffer, a hard drive, a database, an erasable programmable read only memory (EPROM), an electrically erasable programmable read only memory (EEPROM), a read only memory (ROM), a flash memory, a hard disk, a floppy disk, cloud storage, and/or so forth. In some embodiments, the remote computing devices 70 may include one or more hardware-based modules (e.g., DSP, FPGA, ASIC) and/or software-based modules (e.g., a module of computer code stored at the memory and executed at the processor, a set of processor-readable instructions that may be stored at the memory and executed at the processor) associated with executing an application.

The processor of the remote computing device(s) 70 may be operable to control operation of the output of the remote computing device(s) 70. The output may be a television, a liquid crystal display (LCD) monitor, a cathode ray tube (CRT) monitor, speaker, tactile output device, and/or the like. In some embodiments, the output may be an integral component of the remote computing devices 70. Similarly stated, the output may be directly coupled to the processor. For example, the output may be the integral display of a tablet and/or smart phone. In some embodiments, an output module may include, for example, a HDMI connector, a VGA connector, a USB connector, a tip ring sleeve (TRS) connector, and/or any other suitable connector operable to couple the remote computing device(s) 70 to the output.

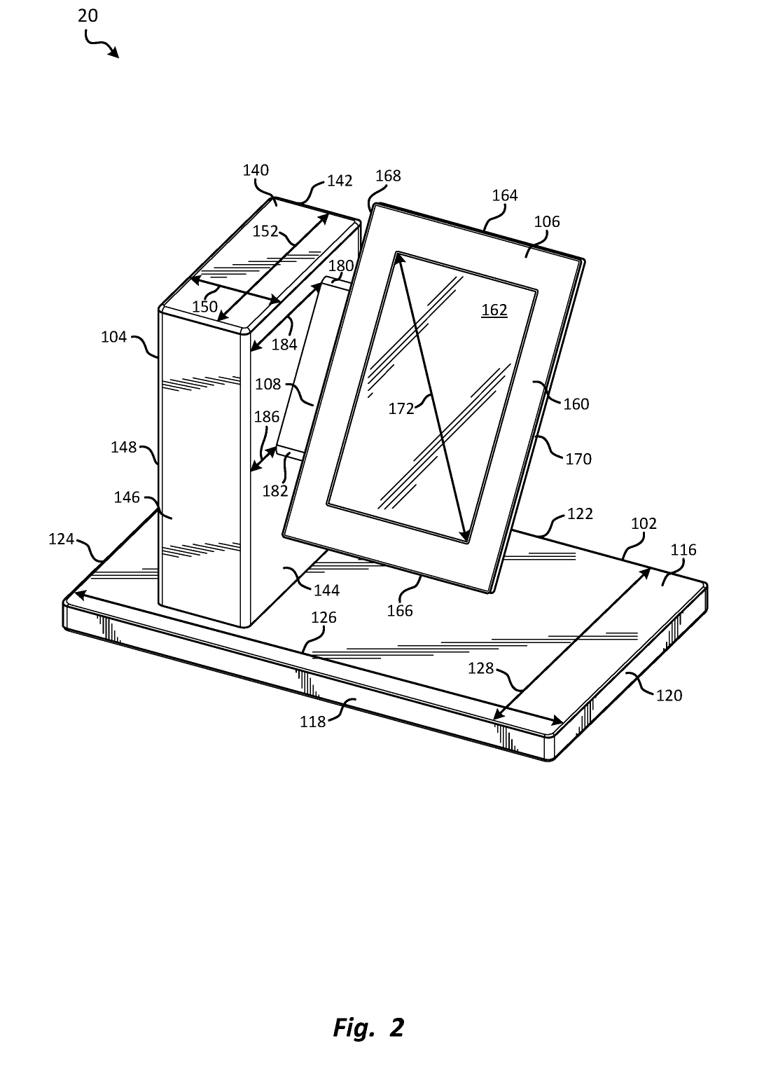

FIG. 2 illustrates a perspective view of the data management device 20. The data management device 20 may be configured to generate and store reports based on data received from instruments 30 and other sources, issue commands to various connected entities, or operatively couple legacy entities and devices to the network 60. The data management device 20 may include a base 102, a pillar 104, a touchscreen user interface 106, and an arm 108.

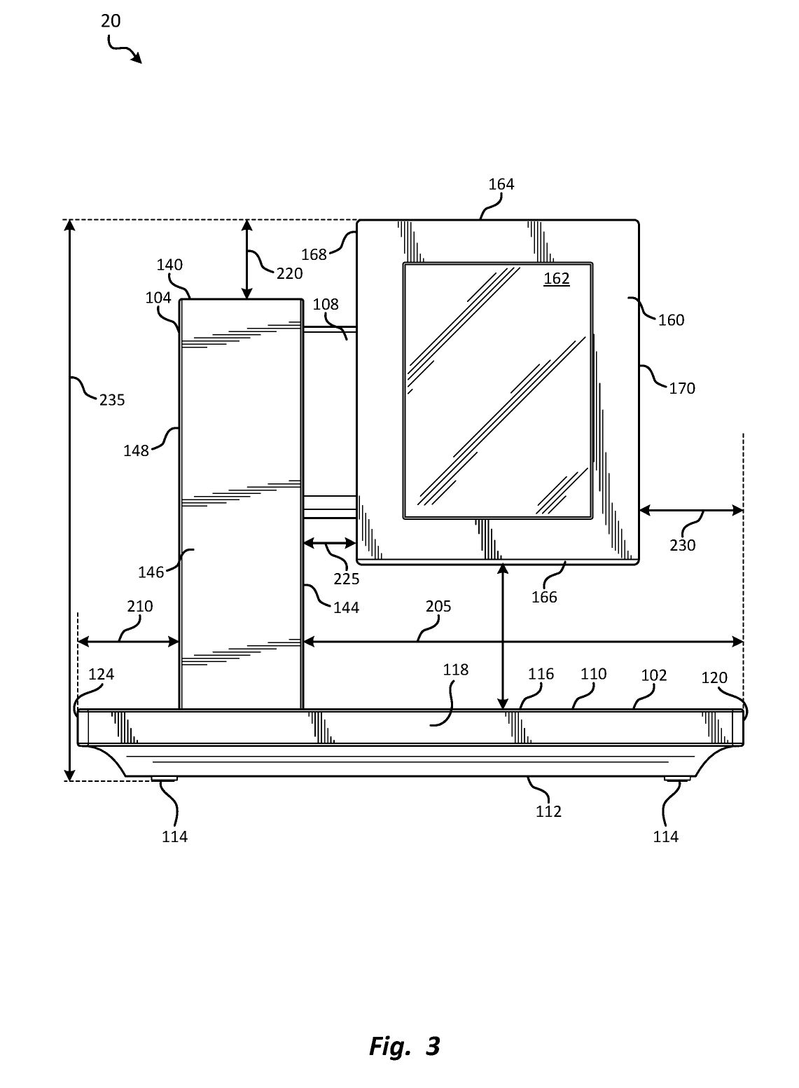

The base 102 may be configured to support the other elements of the data management device 20 while the device 20 is positioned on a flat surface. The base 102 may include an upper portion 110 and a lower portion 112. As shown in more detail in FIG. 3, the upper portion 110 is positioned above the lower portion 112. The lower portion 112 tampers inwardly and downwardly from the side surfaces of the upper portion 110. The lower portion 112 may include feet 114 to interface with corresponding surfaces.

Returning to FIG. 2, the upper portion 110 of the base 102 may include a top surface 116 and side surfaces 118, 120, 122, 124 extending between the top surface 116 and the lower portion 112 of the base 102. The side surface 122 may be positioned opposite the side surface 118 and the side surface 124 may be positioned opposite the side surface 120.

The pillar 104 may be configured to support the touchscreen user interface 106 and/or the arm 108. The pillar 104 may also include a plurality of data ports (e.g. USB ports) to operatively couple the data management device 20 to other devices such as, for example, instruments 30, legacy devices, other types of device, or various combinations thereof.

The pillar 104 extends upwardly from the base 102. The pillar 104 includes a top wall 140 positioned away from the top surface 116 of the base 102. In some examples, the top wall 140 is parallel to the top surface 116. The pillar 104 includes a first sidewall 142, a second sidewall 144, a third sidewall 146, and a fourth sidewall 148 extending between the top wall 140 and the top surface 116. In some examples, the first sidewall 142 includes the data ports. The touchscreen user interface 106 and the arm 108 may be coupled to the second sidewall 144 of the pillar 104.

The top wall 140 of the pillar 104 may define a third dimension 150 extending in the first direction and a fourth dimension 152 extending in the second direction different from the first direction. In some examples, the third dimension 150 extends in the same direction as the first dimension 126 and the fourth dimension 152 extends in the same direction as the second dimension 128. In some examples, the second direction is orthogonal to the first direction. The third dimension 150 may be less than the fourth dimension 152. In some examples, the longer dimension of the top surface 116 (e.g., the first dimension 126) may be perpendicular to the longer dimension of the top wall 140 (e.g., the third dimension 150).

The touchscreen user interface 106 may be configured to output information to a user of the data management device 20 and/or to receive inputs from a user of the data management device 20. As used in this disclosure, the touchscreen user interface may refer to an entire user interface device. For example, the touchscreen user interface 106 may include a housing 160 and screen 162. The screen 162 may be a touchscreen configured to output information and receive inputs. In other examples, the touchscreen user interface 106 may include another input devices such as a keyboard, a mouse, or other cursor moving device.

The touchscreen user interface 106 may include a superior surface 164 positioned at the top of the touchscreen user interface 106 and an inferior surface 166 positioned opposite the superior surface 164. The touchscreen user interface 106 may include a medial surface 168 extending between the superior surface 164 and the inferior surface 166 and a lateral surface 170 positioned opposite the medial surface 168.

The touchscreen user interface 106 may be angled relative to other portions of the data management device 20 such that touchscreen user interface 106 forms a viewing angle. The touchscreen user interface 106 may be configured to be water resistant or water proof. In some lab settings, water may come in contact with the data management device 20. The touchscreen user interface 106 and the other components of the data management device 20 may be water resistant such that the components (e.g., processor, memory, other circuitry) housed in an interior cavity of the data management device 20 are not damaged by water contacting the outer shell of the data management device 20. The touchscreen user interface 106 may be configured such that a user with a gloved hand may be able to input commands via the touchscreen user interface 106.

The touchscreen user interface 106 may define a fifth dimension 172 extending diagonally across the screen of the touchscreen user interface 106. In some examples, the fifth dimension 172 may range between 6.7 inches and 7.3 inches, 6.8 inches and 7.2 inches, 6.9 inches and 7.1 inches, or may be about 7.0 inches.

The arm 108 may be configured to couple the touchscreen user interface 106 to the pillar 104. The arm 108 may suspend the touchscreen user interface 106 above the base 102. The arm 108 may be in contact with or coupled to the second sidewall 144 of the pillar 104. The arm 108 may be angled relative to the first sidewall 142 of the pillar 104. The arm 108 may form an elongated member that extends from a first end 180 to a second end 182. The first end 180 may cooperate with the third sidewall 146 to form a first distance 184. The second end 182 may cooperate with the third sidewall 146 to form a second distance 186 different from the first distance 184. In some examples, the first distance 184 may be greater than the second distance 186.

FIG. 3 illustrates a front elevation view of the data management device 20. FIG. 3 illustrates relative dimensions and positioning of various components of the data management device 20.

The pillar 104 may be positioned to be offset from the center of the base 102 in the first direction. For example, the second sidewall 144 of the pillar 104 may cooperate with a second side surface 120 of the base 102 to define a third distance 205 in the first direction. The fourth sidewall 148 of the pillar 104 may cooperate with a fourth side surface 124 of the base 102 to define a fourth distance 210 different from the third distance 205. In some examples, the third distance 205 may be greater than the fourth distance 210. The second sidewall 144 and the second side surface 120 may cooperate to define a first region of the top surface 116 of the base 102 above which the touchscreen user interface 106 is suspended. The first region may be larger than a second region of the top surface 116 of the base 102 defined by the fourth sidewall 148 and the fourth side surface 124.

The touchscreen user interface 106 may be positioned such that it is entirely above the base 102. The touchscreen user interface 106 may be suspended above the base 102 by the pillar 104 and the arm 108 such that a fifth distance 215 is defined between the top surface 116 of the base 102 and the inferior surface 166 of the touchscreen user interface 106. The superior surface 164 of the touchscreen user interface 106 is positioned above the top wall 140 of the pillar 104 such that a sixth distance 220 is defined between the top wall 140 and the superior surface 164. The medial surface 168 of the touchscreen user interface 106 may be positioned to be spaced apart from the second sidewall 144 of the pillar 104 such that a seventh distance 225 is defined between the second sidewall 144 and the medial surface 168. The lateral surface 170 of the touchscreen user interface 106 may be positioned between the second sidewall 144 of the pillar 104 and the second side surface 120 of the base 102 such that an eighth distance 230 is defined between the second side surface 120 and the lateral surface 170. In some examples, the eighth distance 230 may be greater than the seventh distance 225.

The data management device 20 may define a sixth dimension 235 extending in a third direction between the superior surface 164 of the touchscreen user interface 106 and the feet 114 of the base 102. In some examples, the third direction may be orthogonal to both the first direction and the second direction. In some examples, the sixth dimension 235 may range between 10.7 inches and 11.3 inches, 10.8 inches and 11.2 inches, 10.9 inches and 11.1 inches, or may be about 11.0 inches.

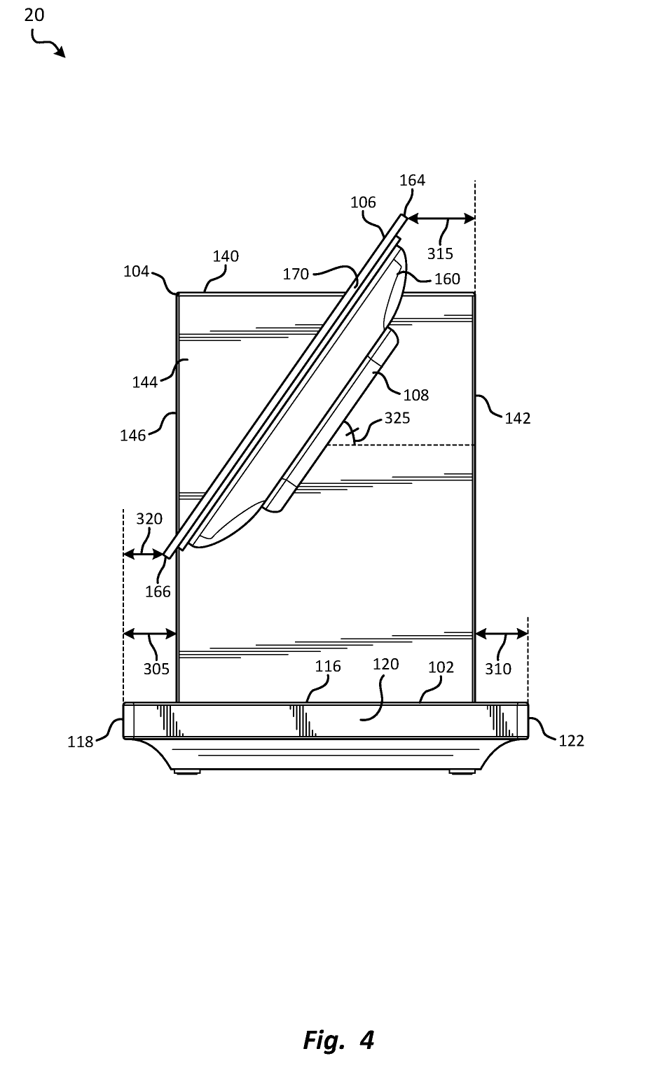

FIG. 4 illustrates a side elevation view of the data management device 20. FIG. 4 illustrates relative dimensions and positioning of various components of the data management device 20.

The pillar 104 may be positioned to be centered on the base 102 in the second direction. For example, the third sidewall 146 of the pillar 104 may cooperate with a first side surface 118 of the base 102 to define a ninth distance 305 between the first side surface 118 and the third sidewall 146. The first sidewall 142 of the pillar 104 may cooperate with the third side surface 122 of the base 102 to define a tenth distance 310 between the third side surface 122 and the first sidewall 142. In some examples, the ninth distance 305 is equal to tenth distance 310. In other examples, the ninth distance 305 may be greater than or lesser than the tenth distance 310.

The touchscreen user interface 106 may be positioned to be offset from the center of base 102 in the second direction. For example, the touchscreen user interface 106 may positioned to be more forward on the base 102. The superior surface 164 of the touchscreen user interface 106 may be positioned to be between the first sidewall 142 and the third sidewall 146 of the pillar 104. The superior surface 164 may cooperate with the first sidewall 142 to define an eleventh distance 315. The inferior surface 166 of the touchscreen user interface 106 may be positioned between the third sidewall 146 of the pillar 104 and the first side surface 118 of the base 102. The inferior surface 166 may cooperate with the first side surface 118 to define a twelfth distance 320. In some examples, the twelfth distance 320 is less than the combination of the eleventh distance 315 and the tenth distance 310.

The touchscreen user interface 106 and/or the arm 108 define an angle 325 relative to the top wall 140 of the pillar 104. In some examples, the angle 325 is a non-orthogonal angle relative to a plane defined by the top wall 140. The angle 325 may be referred to as a viewing angle of the touchscreen user interface 106, in some instances. The angle 325 may be expressed relative to a number of different features of the data management device 20 such as, for example, the top surface 116, the side surfaces 118, 120, 122, 124, sidewalls 142, 144, 146, 148, or combinations thereof. It should also be appreciated that the distances 184, 186 may be expressed in a form relative to the first sidewall 142 instead of the third sidewall 146.

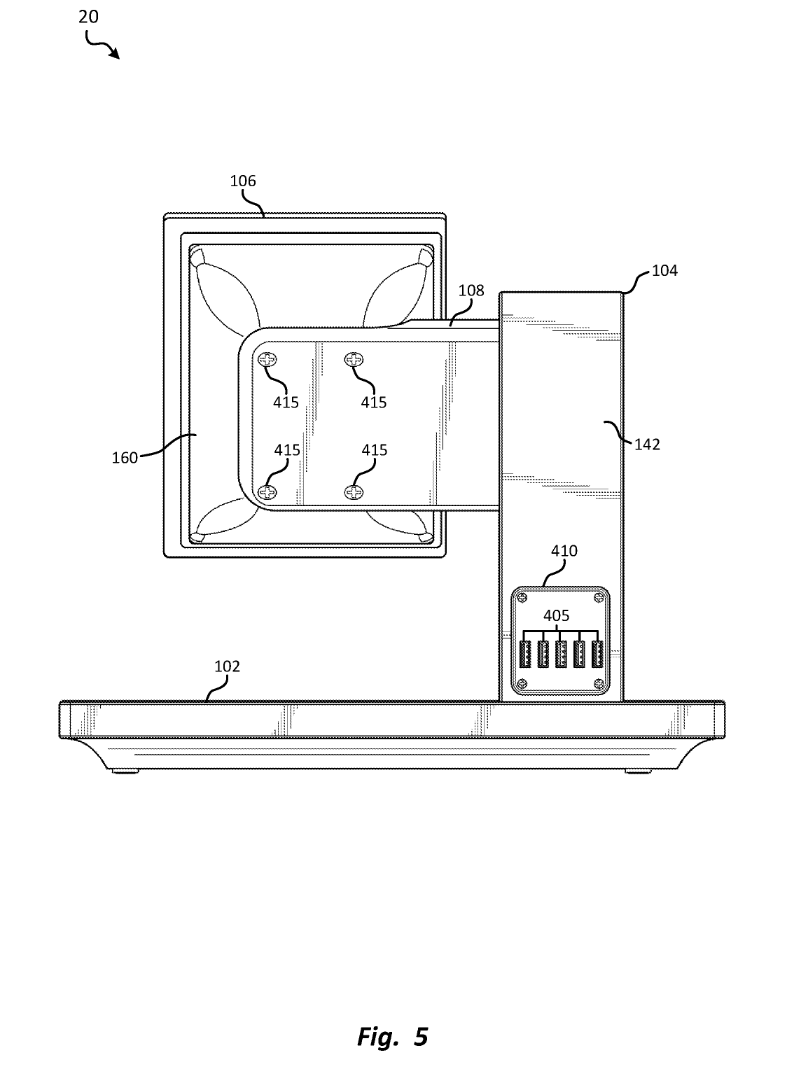

FIG. 5 illustrates a rear elevation view of the data management device 20. The first sidewall 142 may include a number of data ports 405 for transferring data to and from the data management device 20. Each data port 405 may be configured to receive a corresponding cable connector of the same type. For example, the data port 405 may be a female USB port configured to receive a male USB connector on a cable. In some examples, the first sidewall 142 may include a removable plate 410 surrounding the data ports 405. The removable plate 410 may be altered or replaced to accommodate a larger number of data ports 405 than what is illustrated. In some examples, the data ports 405 may be USB ports. In some examples, the data ports 405 may be some other type of port for communicating data via a cable. For example, the data ports may be a VGA port, a DVI port, an HDMI port, an IDE port, a SATA port, an eSATA port, a FireWire port, an Ethernet port, a PS/2 port, a serial port, a RS-232 serial port, a DB-25 serial port, a DE-9 serial port, an S-Video port, a Deutsches Institut für Normung (DIN) port, or combinations thereof.

As shown in more detail in FIG. 5, the arm 108 may contact a back portion of the housing 160 of the touchscreen user interface 106. Fasteners 415, such as screws, may couple the touchscreen user interface 106 to the arm 108.

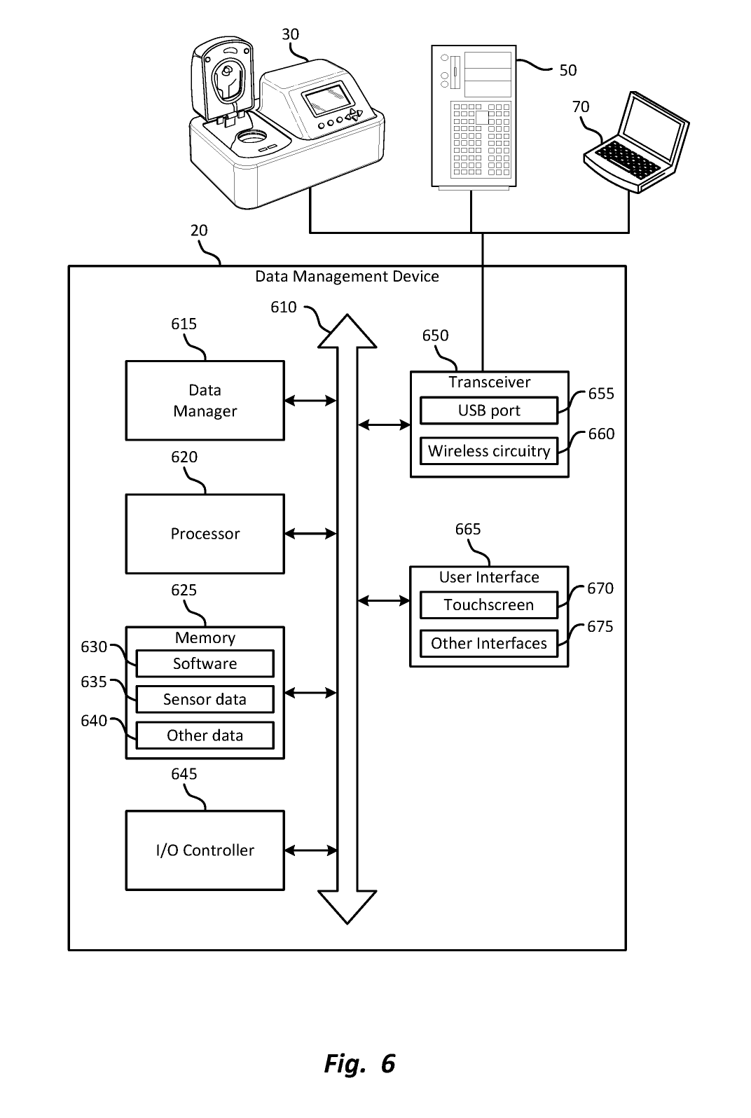

FIG. 6 shows a block diagram of the data management device 20. The data management device 20 may include components for bi-directional voice and data communications including components for transmitting and receiving communications, including data manager 615, processor 620, memory 625, software 630, I/O controller 645, transceiver 650, and user interface 665. These components may be in electronic communication via one or more busses (e.g., bus 610).

In some cases, device 20 may communicate with an instrument 30, a remote storage device, and/or a remote server (e.g., server 50). For example, one or more elements of device 20 may provide a direct connection to a remote server via a direct network link to the Internet via a POP (point of presence). In some embodiments, one element of device 20 (e.g., one or more antennas, transceivers, etc.) may provide a connection using wireless techniques, including digital cellular telephone connection, Cellular Digital Packet Data (CDPD) connection, digital satellite data connection, and/or another connection.

Many other devices and/or subsystems may be connected to one or may be included as one or more elements of the device 20 (e.g., computing device, remote cameras, wireless key fob, wall mounted user interface device, cell radio module, battery, alarm siren, lighting system, and so on). In some embodiments, all of the elements shown in FIG. 6 need not be present to practice the present systems and methods. The devices and subsystems may also be interconnected in different ways from that shown in FIG. 6. In some embodiments, an aspect of the operations of the device 20 may be readily known in the art and are not discussed in detail in this disclosure.

The signals associated with the device 20 may include wireless communication signals such as radio frequency, electromagnetics, LAN, WAN, VPN, wireless network (using 802.11, for example), 345 MHz, Z-WAVE®, cellular network (using 3G and/or Long Term Evolution (LTE), for example), and/or other signals. The radio access technology (RAT) of the device 20 may be related to, but are not limited to, wireless wide area network (WWAN) (GSM, CDMA, and WCDMA), wireless local area network (WLAN) (including BLUETOOTH® and Wi-Fi), WiMAX, antennas for mobile communications, antennas for Wireless Personal Area Network (WPAN) applications (including radio frequency identification devices (RFID) and UWB). In some embodiments, one or more sensors (e.g., motion, proximity, smoke, light, glass break, door, window, carbon monoxide, and/or another sensor) may connect to some element of the device 20 via a network using the one or more wired and/or wireless connections.

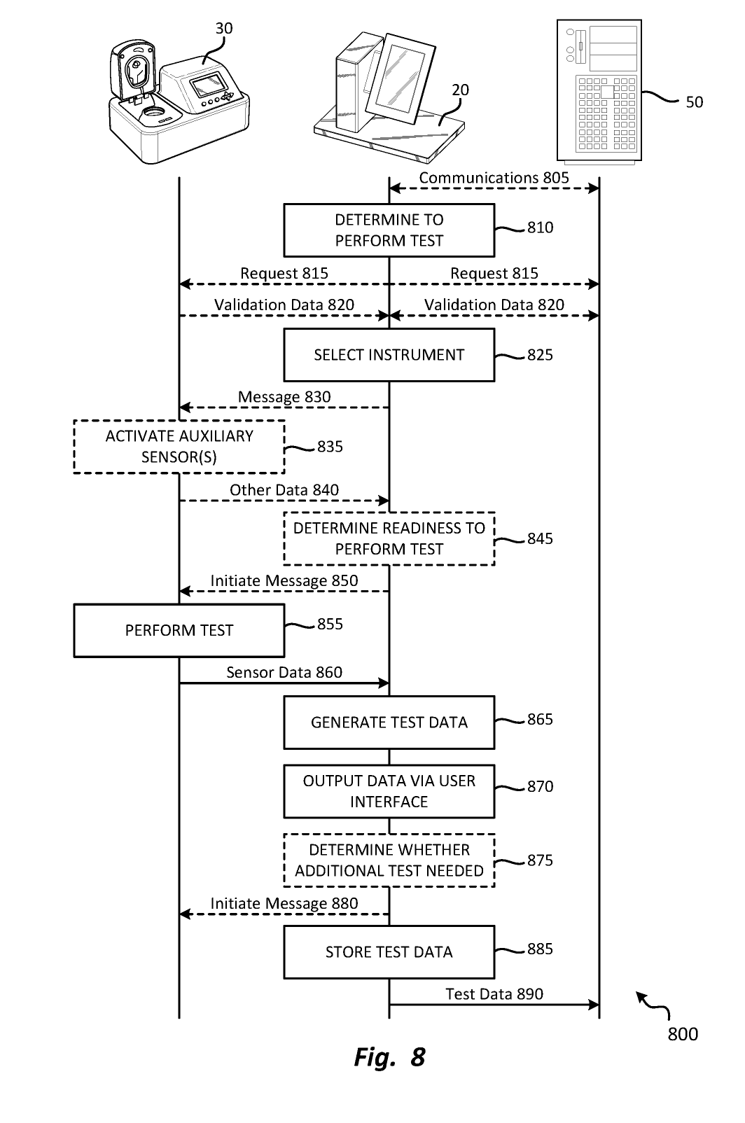

The data manager 615 may be configured to perform the functions described herein. For example, the data manager 615 may be configured to generate reports (e.g., digital quality reports) based on sensor data and other data received from various entities of the lab system 10. In some examples, the data manager 615 may generate reports and execute actions based on data, messages, or commands received from instruments 30, the server 50, or the remote computing devices 70. The data manager 615 may communicate data, messages, or commands to or from one or more other entities in the lab system 10 via network 60 or cables 40. The data manager 615 may also record and report other information regarding the test performed on the sample. Additional features of the data manager 615 are also described herein, especially with reference to FIGS. 8-10.

Processor 620 may include an intelligent hardware device, (e.g., a general-purpose processor, a DSP, a central processing unit (CPU), a microcontroller, an ASIC, an FPGA, a programmable logic device, a discrete gate or transistor logic component, a discrete hardware component, or any combination thereof). In some cases, processor 620 may be configured to operate a memory array using a memory controller. In other cases, a memory controller may be integrated into processor 620. Processor 620 may be configured to execute computer-readable instructions stored in a memory to perform various functions.

Memory 625 may include RAM and ROM. The memory 625 may store computer-readable, computer-executable software 630 including instructions that, when executed, cause the processor to perform various functions described herein. In some cases, the memory 625 may store the sensor data 635 associated with instruments 30 connected to the device 20 and other data 640. In some cases, the memory 625 may contain, among other things, a basic input/output system (BIOS) which may control basic hardware and/or software operation such as the interaction with peripheral components or devices.

Software 630 may include code to implement aspects of the present disclosure, including code to support the device 20. Software 630 may be stored in a non-transitory computer-readable medium such as system memory or other memory. In some cases, the software 630 may not be directly executable by the processor but may cause a computer (e.g., when compiled and executed) to perform functions described herein.

The sensor data 635 may include data generated by sensors of instrument 30 associated with samples. For example, an instrument 30 connected to the data management device 20 may measure various characteristics of a sample. The data associated with those measurements may be referred to as sensor data.

The other data 640 may include any other data associated with the sensor data 635. For example, the other data 640 may include validation data, encryption data, metadata associated with the sensor data, instrument data, workflow data, sample data, testing data, or various combinations thereof. Validation data may indicate whether the instrument 30 complies with applicable best practices, standard operating procedures, or regulations at the time the test was performed. Instrument data may indicate how the instrument 30 was operating at the time of the test. The encryption data may indicate how the data is encrypted. Workflow data may indicate how the test and various other operations and procedures fit into larger lab procedures. Sample data may indicate other information about the sample being tested other than that detectable by the sensors of the instrument 30. Testing data may indicate other information and parameters about the test being performed.

I/O controller 645 may manage input and output signals for device 20. I/O controller 645 may also manage peripherals not integrated into device 20. In some cases, I/O controller 645 may represent a physical connection or port to an external peripheral. In some cases, I/O controller 645 may utilize an operating system such as iOS®, ANDROID®, MS-DOS®, MS-WINDOWS®, OS/2®, UNIX®, LINUX®, or another known operating system. In other cases, I/O controller 645 may represent or interact with a modem, a keyboard, a mouse, a touchscreen, or a similar device. In some cases, I/O controller 645 may be implemented as part of a processor. In some cases, a user may interact with the device 20 via I/O controller 645 or via hardware components controlled by I/O controller 645.

Transceiver 650 may communicate bi-directionally, via one or more antennas, wired, or wireless links as described above. For example, the transceiver 650 may represent a wireless transceiver and may communicate bi-directionally with another wireless transceiver. The transceiver 650 may also include a modem to modulate the packets and provide the modulated packets to the antennas for transmission, and to demodulate packets received from the antennas. The transceiver 650 may communicate bi-directionally with the instruments 30, the server 50, the remote computing devices 70 or combinations thereof.

The transceiver 650 may include a USB port 655, wireless network circuitry 660, other network components or ports, or combinations thereof. The USB port 655 may be an example of the data ports 405 described with reference to FIG. 5. The wireless network circuitry 660 may be configured to establish a wireless communication link 62 via the network 60. The other network components or ports may be any other type of communication circuitry to establish communications (either wired or wireless) between the data management device 20 and the other entities of the lab system 10. For example, the other network components may include components related to VGA, DVI, HDMI, IDE, SATA, eSATA, FireWire, Ethernet, PS/2, a serial connections, a RS-232 serial connection, a DB-25 serial connection, a DE-9 serial connection, an S-Video connection, a DIN connection, Wi-Fi, LTE, 3G, Bluetooth, Bluetooth Low Energy, WLAN, WiGig, or combinations thereof.

User interface 665 may enable a user to interact with the device 20. In some embodiments, the user interface 665 may include an audio device, such as an external speaker system, an external display device such as a display screen, and/or an input device (e.g., remote control device interfaced with the user interface 665 directly and/or through the I/O controller module).

The user interface 665 may include a touchscreen 670 or other interface devices 675. The touchscreen 670 may be an example of the touchscreen user interface 106 described with reference to FIGS. 2-5. The touchscreen 670 may be configured to output information to the user and receive inputs from the user. The other interface devices 675 may include audio interfaces such as speakers, visual interfaces such as visual displays, screens, lights, or other indicators, input devices such as keyboards, mice, touchpads, or other input devices, or combinations thereof.

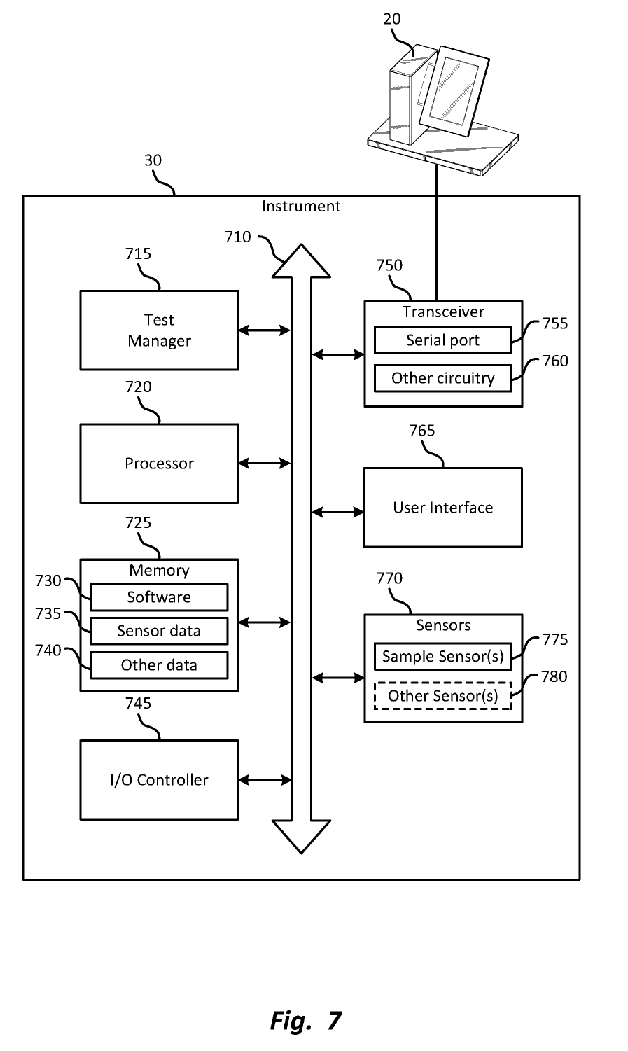

FIG. 7 shows a block diagram of an instrument 30. The instrument 30 may include components for bi-directional voice and data communications including components for transmitting and receiving communications, including test manager 715, processor 720, memory 725, software 730, I/O controller 745, transceiver 750, user interface 765, and sensor(s) 770. These components may be in electronic communication via one or more busses (e.g., bus 710).

In some cases, instrument 30 may communicate with the data management device 20, a remote storage device, a remote server (e.g., server 50), and/or remote computing devices 70. For example, one or more elements of the instrument 30 may provide a direct connection to a remote server via a direct network link to the Internet via a POP (point of presence). In some embodiments, one element of the instrument 30 (e.g., one or more antennas, transceivers, etc.) may provide a connection using wireless techniques, including digital cellular telephone connection, Cellular Digital Packet Data (CDPD) connection, digital satellite data connection, and/or another connection.

The instrument 30 may be a device operable to measure one or more characteristics of a sample. For example, the instrument 30 may be configured to measure a water-based parameter of a food sample taken during a food manufacturing process. Features of the instrument 30 may be functionally and/or structurally similar to the data management system (e.g., processor, memory, I/O controller, transceiver, user interface). As such, full descriptions of these features are not repeated here. Features unique to the instrument 30 are discussed below.

The test manager 715 may be configured to perform the functions described herein. For example, the test manager 715 may be configured to perform a test on a sample to determine a number of characteristics of the sample. In some examples, the test manager 715 executes the test based on a command received from the data management device 20, the server 50, or the remote computing device 70. The test manager 715 may communicate sensor data to one or more other entities in the lab system 10 via the network 60 or a cable 40. The test manager 715 may also record and report other information regarding the test performed on the sample. Additional features of the test manager 715 are also described herein, especially with reference to FIGS. 8-10.

Transceiver 750 may communicate bi-directionally, via one or more antennas, wired, or wireless links as described above. For example, the transceiver 750 may communicate bi-directionally with the data management device 20. In some examples, the transceiver 750 may communicate directly with other instruments 30, the server 50, the remote computing devices 70 or combinations thereof. However, in some embodiments, the data management device 20 connected to the instrument 30 serves as a gateway device for communications to and from the instrument 30. For example, the all communications between a particular instrument 30 and other entities of the lab system 10 may pass through the data management system.

The transceiver 750 may include a serial port 755 and/or other network components or ports 760. The serial port 755 may be an example of a nine pin serial port. In some examples, the serial port 755 is a non-USB serial port. The other circuitry 760 may be configured to establish a wireless communication link 62 via the network 60. The other network components or ports may be any other type of communication circuitry to establish communications (either wired or wireless) between the instrument 30 and the other entities of the lab system 10. For example, the other network components may include components related to VGA, DVI, HDMI, IDE, SATA, eSATA, FireWire, Ethernet, PS/2, a serial connections, USB connections, a RS-232 serial connection, a DB-25 serial connection, a DE-9 serial connection, an S-Video connection, a DIN connection, Wi-Fi, LTE, 3G, Bluetooth, Bluetooth Low Energy, WLAN, WiGig, or combinations thereof.

User interface 765 may enable a user to interact with the instrument 30. In some embodiments, the user interface 765 may include an audio device, such as an external speaker system, an external display device such as a display screen, and/or an input device (e.g., remote control device interfaced with the user interface 765 directly and/or through the I/O controller module). The user interface 765 may include a touchscreen. The touchscreen may be configured to output information to the user and receive inputs from the user. Other interface devices may include audio interfaces such as speakers, visual interfaces such as visual displays, screens, lights, or other indicators, input devices such as keyboards, mice, touchpads, or other input devices, or combinations thereof.

Sensor(s) 770 may be configured to measure a parameter or characteristic of a sample being tested by the instrument 30. In some examples, the instrument 30 may include at least two types of sensors, sample sensor(s) 775 and other sensor(s) 780.

Sample sensor(s) 775 may be configured to measure characteristics about the sample being tested. Sample sensors 775 may include a water activity sensor, a moisture sensor, a pH sensor, a near infrared sensor, a color sensor, a light sensor, a salt sensor, a temperature sensor, an oxygen sensor, a weight sensor, or combinations thereof. In some examples, the instrument 30 may include a single sample sensor 775. In other examples, the instrument 30 may include multiple sample sensors 775.

Other sensor(s) 780 may be configured to identify other parameters and characteristics related to a test being performed on a sample. The other sensor(s) 780 may include proximity sensors, environmental condition sensors, or various combinations thereof. In some examples, a proximity sensor may identify a position of a sample relative to the sample sensors 775. In some examples, a proximity sensor may identify a position of a sample sensor 775 or other sensor 780 relative to a portion of a housing of the instrument 30. In some examples, a proximity sensor may identify that an enclosure to hold a sample is secured.

FIG. 8 illustrates an embodiment of a communication scheme 800 between various entities of the lab system 10. The communication scheme 800 depicts examples of communications and operations performed by the data management device 20, instruments 30, and the server 50. In some instances, the communication scheme 800 may include communications with remote computing devices 70 as well. For example, the communications to and from the server 50 or the operations performed by the server 50 may instead be performed, at least in part, by a remote computing device 70.

As part of the communication scheme 800, the server 50 and the data management device 20 may exchange communications 805. Communications 805 may include any types of data, messages, or commands. For example, the server 50 may transmit a command instructing the data management device 20 to execute a particular test. In other examples, the server 50 may transmit a request for data from the data management device 20, such as data, sensor data, or reports. The data management device 20 may transmit such data to the server 50. In some examples, the data management device 20 may transmit a message to the server 50 requesting particular information, such as additional information related to the test to be executed or information related to the instruments operatively coupled to the data management device 20.

At block 810, the data management device 20 may determine to perform a test using one of the instruments 30 connected to the data management device 20. The determination by the data management device 20 may be based on the communications 805 received from the server 50 or a remote computing device 70. The determination by the data management device 20 may be based on data input into a user interface of the data management device 20, for example by a technician using the device 20. At part of the determination, the data management device 20 may identify which type of instrument 30 to perform the test. In some cases, the data management device 20 may be connected to several types of equipment, including lab equipment. For example, the data management device 20 may be operatively coupled to both a water activity meter and a moisture analyzer simultaneously.

At block 825, after identifying the type of equipment, the data management device 20 may select a specific instrument 30 operatively coupled to the device 20 to perform the test. Multiple instruments 30 of the same type may be coupled to the data management device 20. As such, the data management device 20 may select a specific instrument 30 to perform the test based on various characteristics and data associated with the instruments in questions.

The data management device 20 may identify a group of instruments from the total number of instruments 30 that are operatively coupled to the device 20 and are capable of performing the requested test. The data management device 20 may transmit a request 815 for additional data associated with each instrument in the group of instruments. The request 815 may include information asking for additional information about the instruments 30. Such additional information may include validation data 820. For each instrument 30 in question, the validation data 820 may indicate a maintenance schedule, the last performed maintenance, a current calibration of the instrument, whether the instrument 30 is currently functioning properly, other information about the operability of the instrument 30, other information about the compliance of the instrument 30 with relevant workflows, rules, regulations, standard operating procedures, or combinations thereof. In some examples, the validation data 820 may be stored at the instrument 30 in question, stored at the data management device 20, or stored at the server 50 or remote computing devices 70. In examples where the validation data 820 is stored at the device 20, the data management device 20 may not transmit the request 815.

The data management device 20 may select an instrument 30 from the group of instruments based on the validation data. For example, the device 20 may select instrument 30 based on operability parameters (e.g., calibration and is the instrument working) and compliance parameters. In the event that none of the connected instruments 30 in the group of instruments capable of performing the requested test can perform the request test, either due to operability reasons or compliance reasons, the device 20 may output a message to the user of the lab system 10 that none of the connected instruments are able to execute the request test. In some examples, the data management device 20 may forward the request to perform a test to another device 20 in the lab system 10 based on not having a connected device capable of performing the requested test.

After selecting instrument 30 to perform the requested test, the data management device 20 may transmit a message 830 to the selected instrument 30 with instructions to perform the requested test. The message 830 may include test parameters indicating what type of test is to be performed and the relevant characteristics of the test. In some examples, a sample of some type may be positioned, either by a person or a machine, in or on the instrument 30. In some machine placement examples, the device 20 may generate and transmit commands related to the placement of the sample.

Upon receiving the message 830, the selected instrument 30 may initiate protocols to execute the request test. Such protocols may include determining whether a sample is in position or whether the instrument 30 is in a proper state to perform the requested test. In some instances, at block 835, the instrument 30 may activate one or more of its auxiliary sensors to determine whether an instrument is ready to perform the test. For example, the instrument 30 may activate one or more proximity sensors to determine whether a primary sensor (e.g., sample sensor) is in a correct position, determine whether a food sample is in a correct position, determine a relative position of the primary sensor to a housing of the instrument 30, determine a relative position of the primary sensor to the food sample, or combinations thereof. In other examples, the instrument 30 may measure environmental conditions or other conditions to determine whether it is appropriate to perform the requested test.

The instrument 30 may transmit other data 840 to the data management device 20. The other data 840 may include data generated by the auxiliary sensor(s). For example, the other data 840 may be proximity data generated by a proximity sensor. In other examples, the other data 840 may be calibration data generated by internal sensors and system checks performed by the instrument 30. In other examples, the other data 840 may be environmental data or other types of data. In some examples, the data management device 20 may transmit the other data 840 or validation data 820 or other types of data to the server 50 to be stored.

At block 845, the data management device 20 may determine whether the instrument 30 is ready to perform the requested test. Such a determination may be based on the other data 840 received from the instrument 30. In some examples, the determination may include determining that a position of a sensor relative to another component or object such as, for example, the sample or the housing of the instrument 30. Upon determining that the instrument 30 is ready to execute, the data management device 20 may transmit an initiate message 850 to the instrument instructing to perform the requested test. In some examples, the data management device 20 may output the other data 840 to a user via its user interface. In such an example, the data management device 20 may generate the initiate message 850 based on input received from the user. In some examples, the input received from the user may be received in response to outputting the other data 840.

In some examples, a test may be initiated locally at the instrument 30 and the associated data may be “pushed” to the data management device 20 without the device 20 initiating the test. In such examples, the functions and messages described with relationship to blocks 830, 835, 840, 845, 850 may not be performed by the lab system 10.

At block 855, the instrument 30 may perform the requested test. Performing the requested test may include measuring, by the instrument 30, one or more characteristics of the sample. The instrument 30 may generate sensor data 860 based on the measurements performed by a primary sensor (e.g., a sample sensor) of the instrument. In some examples, the sensor data 860 may include additional data related to the test, other than the output of the primary sensor(s). For example, the sensor data 860 may include validation data, proximity data, calibration data, workflow data, compliance data, other data, or various combinations thereof.

At block 865, the data management device 20 may generate test data based on various data collected prior to, during, or after performing the requested test. In some examples, the test data may take the form of a digital report (e.g., digital lab report). The report may combine sensor data and various other kinds of data. For example test data may include a name of a user of the data management device 20, a time stamp, a data stamp, sensor data such as a reading from a instrument, identification numbers for each device or entity that participates in the requested test, data related to the sample such as batch number, lot number, product name, etc., operating limits, workflow data, calibration data, validation data, or combinations thereof.

In some examples, the data management device 20 may encrypt the test data for transport as part of generating the test data. Encryption of the test data may be accomplished by any number of encryption techniques including, but not limited to, a private key/public key method or a hashing method.

In some examples, the data management device 20 may select a mode of operation associated with generating the test data. For example, the data management device 20 may include a strict mode of operation and non-strict mode of operation. In the strict mode of operation the acceptable tolerances for test data and compliance with regulations may be more rigorous than in the non-strict mode. For example, in a strict mode, the data management device may compare the test data to all relevant regulations and validation standards. In the strict mode, the data management device 20 may label test data as unacceptable if any of the test data fails to satisfy all regulations and rules. In the non-strict mode, the data management device 20 may have more relaxed tolerances such that more test data may be labeled as acceptable. The non-strict mode may be used for processes that are governed by standard operating procedures rather than governmental regulations.

At block 870, the data management device 20 may output the test data via a user interface of the device 20. The data management device 20 output information or visual graphics prompting a user of the device 20 to review and/or approve the test data.

At block 875, the data management device 20 may determine whether an additional test is needed. Such a determination may be based on comparing the test data of test to a number of quality thresholds. If the test data fails to satisfy some or all of the quality thresholds, the device 20 may generate a request to perform an additional test. In some examples, the data management device 20 may determine whether an additional test is needed automatically, without receiving input from a user. If an additional test is needed, the data management device 20 may generate and transmit another initiate message 880 similar to the initiate message 850.

In some examples, the data management device 20 may compare a number of failed tests to a failed test threshold. If the number of failed tests satisfies the threshold, the data management device 20 may reject the test data or indicate the test data as unacceptable. In some examples, the data management device 20 may output data informing a user about the numerous failed tests satisfying the threshold.

At block 885, the data management device 20 determines whether to store the test data or the reports generated from test data. The determination may be based on whether the test data satisfied some or all of the quality thresholds. In some examples, upon determining to store the test data, the device 20 may encrypt the test data.

The data management device 20 may transmit the test data 890 to the server 50 or remote computing device 70. In some examples, the transmission of test data 890 may not be based on the determination found in block 885. Once the server 50 or the remote computing device 70 receives the test data 890, a remote computing device 70 may perform various operations based on the test data 890. In some examples, such operations may be performed by a user utilizing the remote computing device 70. For example, a remote computing device 70 may be configured to output the test data 890 via a user interface, create reports based on the test data 890, manipulate or plot the test data 890, manage user accounts of the lab system 10, set operating limits, regulatory controls, workflows, standard operating procedures, or other parameters associated with the test data 890 and the requested test from which the test data 890 came, manage corrective actions, or combinations thereof. In some examples, the remote computing device 70 may provide an interface to a user via a web browser.

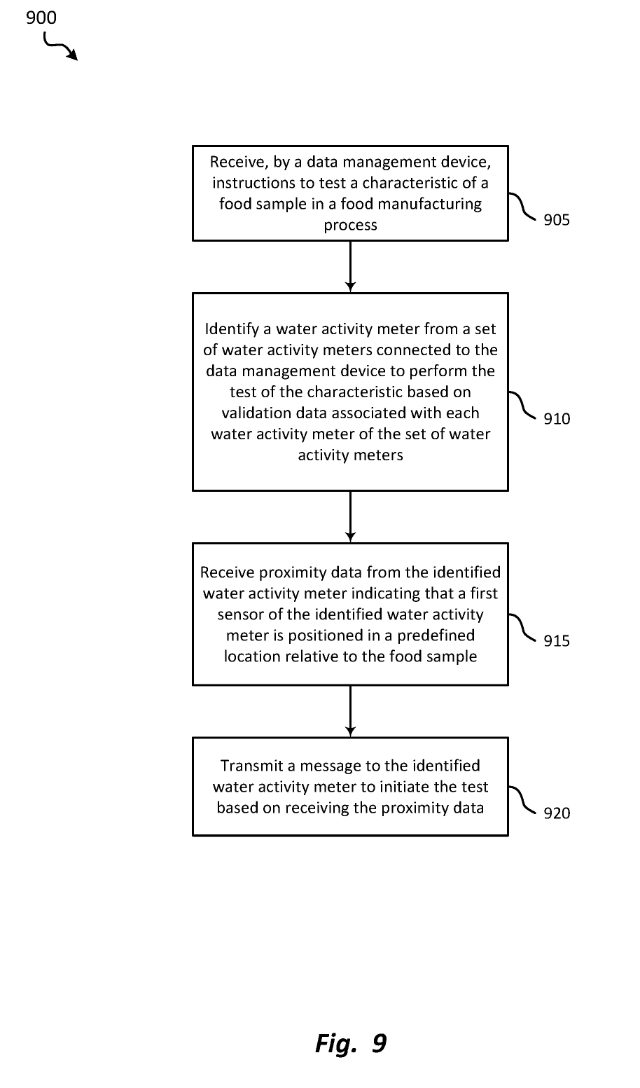

FIG. 9 illustrates a flowchart illustrating a method 900 for systems, devices, and method for managing data in accordance with various aspects of the present disclosure. The operations of method 900 may be implemented by a lab system 10 or its components as described herein. For example, the operations of method 900 may be performed by a data management device 20 the as described with reference to FIGS. 1 through 6 and 8. In some examples, a data management device 20 may execute a set of codes to control the functional elements of the device to perform the functions described below. Additionally or alternatively, the data management device 20 may perform aspects of the functions described below using special-purpose hardware.

At block 905 the data management device 20 may receive instructions to test a characteristic of a food sample in a food manufacturing process. In certain examples, aspects of the operations of block 905 may be performed by a data manager 615 as described with reference to FIG. 6.

At block 910 the data management device 20 may identify a water activity meter from a plurality of water activity meters connected to the data management device to perform the test of the characteristic based at least in part on validation data associated with each water activity meter of the plurality of water activity meters. In certain examples, aspects of the operations of block 910 may be performed by a data manager 615 as described with reference to FIG. 6.

At block 915 the data management device 20 may receive proximity data from the identified water activity meter indicating that a first sensor of the identified water activity meter is positioned in a predefined location relative to the food sample. In certain examples, aspects of the operations of block 915 may be performed by a data manager 615 as described with reference to FIG. 6.

At block 920 the data management device 20 may transmit a message to the identified water activity meter to initiate the test based at least in part on receiving the proximity data. In certain examples, aspects of the operations of block 920 may be performed by a data manager 615 as described with reference to FIG. 6.

In some instances, the data management device 20 may perform other functions as part of method 900. For example, the data management device 20 may determine a position of the food sample relative to the first sensor based on the proximity data received from a second sensor associated with the identified water activity meter. In other examples, the data management device 20 may receive, from the first sensor of the identified water activity meter, sensor data indicating a parameter of the food sample based on the water activity meter performing the test. In other examples, the data management device 20 may output the sensor data via a touchscreen user interface of the data management device.

In other examples, the data management device 20 may output an indication of the proximity data via a touchscreen user interface of the data management device. In other examples, the data management device 20 may receive an input command from a user to initiate the test via the touchscreen user interface based on outputting the indication. In other examples, the data management device 20 may compare the validation data of each water activity meter, where identifying the water activity meter is based on the comparison. In other examples, the data management device 20 may identify a water activity meter from a set of water activity meters connected to the data management device to perform the test of the characteristic based on validation data associated with each water activity meter of the set of water activity meters. In some cases, the validation data may include information indicating a maintenance report associated with the water activity meter and a calibration report associated with the water activity meter. In some cases, the validation data may include information indicating whether a validation procedure associated with the water activity meter has been implemented.

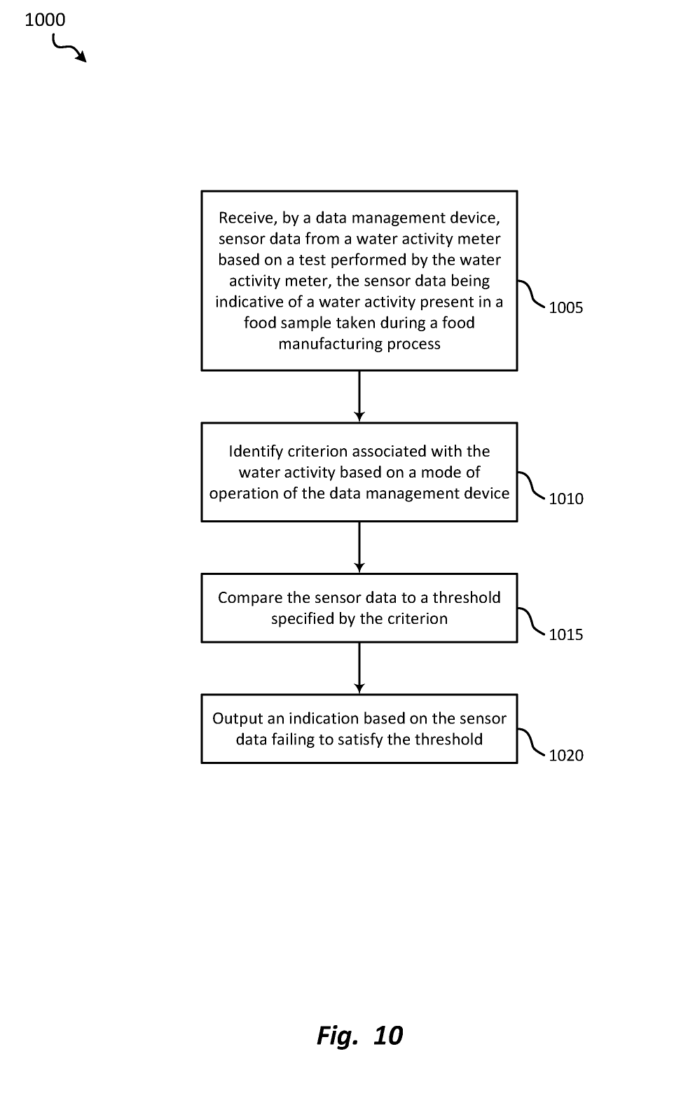

FIG. 10 illustrates a flowchart illustrating a method 1000 for systems, devices, and method for managing data in accordance with various aspects of the present disclosure. The operations of method 1000 may be implemented by a lab system 10 or its components as described herein. For example, the operations of method 1000 may be performed by a data management device 20 as described with reference to FIGS. 1 through 6 and 8. In some examples, a data management device 20 may execute a set of codes to control the functional elements of the device to perform the functions described below. Additionally or alternatively, the data management device 20 may perform aspects of the functions described below using special-purpose hardware.

At block 1005 the data management device 20 may receive sensor data from a water activity meter based on a test performed by the water activity meter, the sensor data being indicative of a water activity present in a food sample taken during a food manufacturing process. In certain examples, aspects of the operations of block 1005 may be performed by a data manager 615 as described with reference to FIG. 6.

At block 1010 the data management device 20 may identify criterion associated with the water activity based at least in part on a mode of operation of the data management device. In certain examples, aspects of the operations of block 1010 may be performed by a data manager 615 as described with reference to FIG. 6.

At block 1015 the data management device 20 may compare the sensor data to a threshold specified by the criterion. In certain examples, aspects of the operations of block 1015 may be performed by a data manager 615 as described with reference to FIG. 6.

At block 1020 the data management device 20 may output an indication based at least in part on the sensor data failing to satisfy the threshold. In certain examples, aspects of the operations of block 1020 may be performed by a data manager 615 as described with reference to FIG. 6.

In some instances, the data management device 20 may perform other functions as part of method 1000. For example, the data management device 20 may transmit, automatically, a message to the water activity meter to perform another test based on the sensor data failing to satisfy the threshold. In other examples, the data management device 20 may identify that a number of failed tests does not satisfy a test threshold, where transmitting the message is based on the test threshold not being satisfied. In other examples, the data management device 20 may receive an input command from a user via a touchscreen user interface of the data management device based on outputting the indication. In some cases, the data management device 20 may include a first mode of operation specifying a first set of criteria associated with the water activity and a second mode of operation specifying a second set of criteria associated with the water activity, the second set of criteria being more strict than the first set of criteria.

The detailed description set forth above in connection with the appended drawings describes examples and does not represent the only instances that may be implemented or that are within the scope of the claims. The terms “example” and “exemplary,” when used in this description, mean “serving as an example, instance, or illustration,” and not “preferred” or “advantageous over other examples.” The detailed description includes specific details for the purpose of providing an understanding of the described techniques. These techniques, however, may be practiced without these specific details. In some instances, known structures and apparatuses are shown in block diagram form in order to avoid obscuring the concepts of the described examples.

Information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and components described in connection with this disclosure may be implemented or performed with a general-purpose processor, a digital signal processor (DSP), an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, and/or state machine. A processor may also be implemented as a combination of computing devices, e.g., a combination of a DSP and a microprocessor, multiple microprocessors, one or more microprocessors in conjunction with a DSP core, and/or any other such configuration. An operating system utilized by the processor (or by I/O controller module or another module described above) may be iOS®, ANDROID®, MS-DOS®, MS-WINDOWS®, OS/2C), UNIX®, LINUX®, or another known operating system.

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope and spirit of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations.

As used herein, including in the claims, the term “and/or,” when used in a list of two or more items, means that any one of the listed items can be employed by itself, or any combination of two or more of the listed items can be employed. For example, if a composition is described as containing components A, B, and/or C, the composition can contain A alone; B alone; C alone; A and B in combination; A and C in combination; B and C in combination; or A, B, and C in combination. Also, as used herein, including in the claims, “or” as used in a list of items (for example, a list of items prefaced by a phrase such as “at least one of” or “one or more of”) indicates a disjunctive list such that, for example, a list of “at least one of A, B, or C” means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

In addition, any disclosure of components contained within other components or separate from other components should be considered exemplary because multiple other architectures may potentially be implemented to achieve the same functionality, including incorporating all, most, and/or some elements as part of one or more unitary structures and/or separate structures.

Computer-readable media includes both computer storage media and communication media including any medium that facilitates transfer of a computer program from one place to another. A storage medium may be any available medium that can be accessed by a general purpose or special purpose computer. By way of example, and not limitation, computer-readable media can comprise RAM, ROM, EEPROM, flash memory, CD-ROM, DVD, or other optical disk storage, magnetic disk storage or other magnetic storage devices, or any other medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include compact disc (CD), laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The previous description of the disclosure is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not to be limited to the examples and designs described herein but is to be accorded the broadest scope consistent with the principles and novel features disclosed.

The process parameters, actions, and steps described and/or illustrated in this disclosure are given by way of example only and can be varied as desired. For example, while the steps illustrated and/or described may be shown or discussed in a particular order, these steps do not necessarily need to be performed in the order illustrated or discussed. The various exemplary methods described and/or illustrated here may also omit one or more of the steps described or illustrated here or include additional steps in addition to those disclosed.

Furthermore, while various embodiments have been described and/or illustrated here in the context of fully functional computing systems, one or more of these exemplary embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may permit and/or instruct a computing system to perform one or more of the exemplary embodiments disclosed here.

This description, for purposes of explanation, has been described with reference to specific embodiments. The illustrative discussions above, however, are not intended to be exhaustive or limit the present systems and methods to the precise forms discussed. Many modifications and variations are possible in view of the above teachings. The embodiments were chosen and described in order to explain the principles of the present systems and methods and their practical applications, to enable others skilled in the art to utilize the present systems, apparatus, and methods and various embodiments with various modifications as may be suited to the particular use contemplated.

What is claimed is:

1. A device, comprising:
a pillar extending upward from a base;
a plurality of data ports positioned on a first sidewall of the pillar, each data port being configured to receive data output from a serial port of a water activity meter; and
a touchscreen user interface coupled to a second sidewall of the pillar different from the first sidewall, the touchscreen user interface being angled relative to the first sidewall such that the touchscreen user interface defines a viewing angle, the touchscreen user interface being configured to output the received data and received commands from a user.

2. The device of claim 1, further comprising:
an arm extending from the second sidewall of the pillar configured to suspend the touchscreen user interface above a top surface of the base, wherein the touchscreen user interface is coupled to the arm.

3. The device of claim 2, wherein the arm is angled relative to the first sidewall of the pillar.

4. The device of claim 3, wherein a first distance between a first end of the arm and the first sidewall is less than a second distance between a second end of the arm and the first sidewall.

5. The device of claim 1, wherein the pillar and the touchscreen user interface are positioned completely above the base such that side surfaces of the base are positioned beyond the pillar and the touchscreen user interface.

6. The device of claim 1, wherein:
the base comprises a top surface having a first base dimension extending in a first direction greater than a second base dimension extending in a second direction of the top surface perpendicular to the first direction, and
the pillar comprises a top wall having a first pillar dimension extending in the first direction that is less than a second pillar dimension extending in the second direction.

7. The device of claim 1, wherein the touchscreen user interface extends from a medial surface spaced apart from the second sidewall to a lateral surface positioned between the second sidewall and a first side surface of the base.

8. The device of claim 1, wherein:
the base comprises a top surface extending from a first side surface to a second side surface positioned opposite the first side surface, and
the pillar is positioned on the base such that a first distance between the second sidewall of the pillar and the first side surface of the base is greater than a second distance between a third sidewall of the pillar and the second side surface of the base, wherein the third sidewall is positioned opposite the second sidewall.

9. The device of claim 8, wherein a third distance between the second sidewall and a lateral surface of the touchscreen user interface is less than the first distance.

10. The device of claim 8, wherein:
the base further comprises a third side surface extending between the first side surface and the second side surface and a fourth side surface extending between the first side surface and the second side surface, the fourth side surface being positioned opposite the third side surface, and
a fourth distance between the first sidewall and the third side surface of the base is less than a fifth distance between a superior surface of the touchscreen user interface and the third side surface of the base.

11. The device of claim 8, wherein a sixth distance between a fourth sidewall of the pillar positioned opposite the first sidewall and the fourth side surface of the base is greater than a seventh distance between an inferior surface of the touchscreen user interface and the fourth side surface of the base.

12. The device of claim 1, wherein the viewing angle of the touchscreen user interface is non-orthogonal relative to a top surface of the base.

13. The device of claim 1, wherein the touchscreen user interface extends above a top wall of the pillar.

14. The device of claim 1, wherein the touchscreen user interface defines a lateral surface positioned between the second sidewall of the pillar and a first side surface of the base.

15. The device of claim 1, wherein the touchscreen user interface includes a housing and a screen.

16. The device of claim 1, further comprising:
a processor and a memory configured to communicate data with the water activity meter via at least one of the plurality of data ports.

17. The device of claim 1, wherein the water activity meter is configured to measure a water activity of a food sample in a food manufacturing process.

18. The device of claim 1, wherein the plurality of data ports are a plurality of universal serial bus (USB) ports.

19. A system, comprising:
an instrument configured to measure a parameter of a food sample in a food manufacturing process, the instrument having a serial port configured to output data indicative of the parameter; and
a data management device including:
a pillar extending upward from a base;
a plurality of universal serial data (USB) ports positioned on a first sidewall of the pillar, each USB data port being configured to receive the data output from the serial port of the instrument; and
a touchscreen user interface coupled to a second sidewall of the pillar different from the first sidewall, the touchscreen user interface being angled relative to the first sidewall such that the touchscreen user interface defines a viewing angle, the touchscreen user interface being configured to output the received data and receive commands from a user.

20. The system of claim 19, further comprising:
a cable having a USB cable connector at a first end and a serial cable connector at a second, the cable extending between one of the USB ports of the data management device and the serial port of the instrument.

21. The system of claim 19, wherein the instrument includes a first sensor to measure the parameter of the food sample and a second sensor to identify a position of the food sample relative to the first sensor.

22. The system of claim 19, wherein the instrument is a water activity meter and the parameter is a water activity.

23. The system of claim 19, wherein the instrument is a moisture analyzer and the parameter is moisture parameter.

24. A method, comprising:
receiving, by a data management device, instructions to test a characteristic of a food sample in a food manufacturing process;
identifying a water activity meter from a plurality of water activity meters connected to the data management device to perform the test of the characteristic based at least in part on validation data associated with each water activity meter of the plurality of water activity meters;
receiving proximity data from the identified water activity meter indicating that a first sensor of the identified water activity meter is positioned in a predefined location relative to the food sample; and
transmitting a message to the identified water activity meter to initiate the test based at least in part on receiving the proximity data.

25. The method of claim 24, further comprising:
determining a position of the food sample relative to the first sensor based at least in part on the proximity data received from a second sensor associated with the identified water activity meter.

26. The method of claim 24, further comprising:
receiving, from the first sensor of the identified water activity meter, sensor data indicating a parameter of the food sample based at least in part on the water activity meter performing the test.

27. The method of claim 26, further comprising:
outputting the sensor data via a touchscreen user interface of the data management device.

28. The method of claim 24, further comprising:
outputting an indication of the proximity data via a touchscreen user interface of the data management device.

29. The method of claim 28, further comprising:
receiving an input command from a user to initiate the test via the touchscreen user interface based at least in part on outputting the indication.

30. The method of claim 24, further comprising:
comparing the validation data of each water activity meter, wherein identifying the water activity meter is based at least in part on the comparison.

31. The method of claim 24, wherein the validation data comprises information indicating whether a validation procedure associated with the water activity meter has been implemented.

32. The method of claim 24, wherein the validation data comprising information indicating a maintenance report associated with the water activity meter and a calibration report associated with the water activity meter.

33. A method, comprising:
receiving, by a data management device, sensor data from a water activity meter based on a test performed by the water activity meter, the sensor data being indicative of a water activity present in a food sample taken during a food manufacturing process;
identifying criterion associated with the water activity based at least in part on a mode of operation of the data management device;
comparing the sensor data to a threshold specified by the criterion; and
outputting an indication based at least in part on the sensor data failing to satisfy the threshold.

34. The method of claim 33, further comprising:
transmitting, automatically, a message to the water activity meter to perform another test based at least in part on the sensor data failing to satisfy the threshold.

35. The method of claim 34, further comprising:
identifying that a number of failed tests does not satisfy a test threshold, wherein transmitting the message is based at least in part on the test threshold not being satisfied.

36. The method of claim 33, further comprising:
receiving an input command from a user via a touchscreen user interface of the data management device based at least in part on outputting the indication.

37. The method of claim 33, wherein the data management device includes a first mode of operation specifying a first set of criteria associated with the water activity and a second mode of operation specifying a second set of criteria associated with the water activity, the second set of criteria being more strict than the first set of criteria.
